(19) United States
(12) Patent Application Publication
(10) Pub. No.: US 2024/0424231 A1
(43) Pub. Date: Dec. 26, 2024

(54) INHALATION AND EXHALATION DEVICES

(71) Applicant: Dendro Technologies, Inc., New York, NY (US)

(72) Inventors: Elisha TAL, Nataf (IL); Raz MOHEBAN, Shoshanat Haamakim (IL); Adi WALLACH, New York, NY (US); Michael YAKOBY, Kfar Saba (IL)

(21) Appl. No.: 18/703,345

(22) PCT Filed: Nov. 8, 2022
(86) PCT No.: PCT/US2022/049182

§ 371 Date: Apr. 21, 2024

Related U.S. Application Data
(60) Provisional application No. 63/276,744, filed on Nov. 8, 2021.

(51) Int. Cl. A61M 15/00; A24F 40/05; A24F 42/20

(52) U.S. Cl. CPC A61M 15/0021; A24F 40/05; A24F 42/20; A61M 2205/3306; A61M 2205/8206

(57) ABSTRACT

An inhalation and/or exhalation device comprising: a body comprising an external surface; a non-protruding mouthpiece comprising a mouthpiece bore having an opening at said external surface; and a gas conduit having a proximal end in fluid communication with said mouthpiece bore and a distal end, the device thereby configured to allow a person whose mouth sealingly contacts a portion of said external surface of said body that surrounds said opening to provide fluid communication between the mouth-hole of the person and said opening to inhale a gas into the mouth, and/or exhale a gas from the mouth into said opening of said mouthpiece.

### RELATED APPLICATION

The present application gains priority from U.S. Provisional Patent Application 63/276,744 filed 8 Nov. 2021 which is included by reference as if fully set-forth herein.

### FIELD AND BACKGROUND OF THE INVENTION

The invention, in some embodiments, relates to inhalation devices, exhalation devices and inhalation/exhalation devices. In some embodiments the disclosed devices have a mouthpiece, preferably a non-protruding mouthpiece. In some embodiments, the devices comprise two assemblies that are reversibly mateable, one of the two assemblies comprising a conduit for the passage of a gas such as air.

Inhalation devices are well-known in the field of pulmonary drug delivery, for example, inhalers for pulmonary administration of drugs to treat asthma or dry-powder inhalers. During use, a user inhales through the device to draw air carrying the drug to be inhaled into the mouth.

Exhalation devices are also known, for example a breathing biofeedback device to assist a person in proper breathing as described in US 2015/0011906 A1. During use, a user exhales from the mouth into the device.

There are also known inhalation/exhalation devices in which during use a user both inhales through the device to draw air into the mouth and exhales from the mouth into the device.

Inhalation, exhalation and inhalation/exhalation devices typically include a protruding hollow mouthpiece which is substantially a tube having an outer wall surface and an inner wall surface defining a bore. For use, a person encircles the outer wall surface with their lips to make a seal, and then inhales or exhales through the mouth.

### SUMMARY OF THE INVENTION

The invention, in some embodiments, relates to inhalation and exhalation devices. In some embodiments the disclosed devices have a mouthpiece, preferably a non-protruding mouthpiece. In some embodiments, the devices comprise two assemblies that are reversibly mateable, one of the two assemblies being a conduit for the passage of air.

### Non-Protruding Mouthpiece

According to an aspect of some embodiments of the teachings herein, there is provided an inhalation and/or exhalation device, comprising:
a body comprising an external surface;
a non-protruding mouthpiece having a mouthpiece bore with an opening at the external surface; and
a gas conduit having a proximal end in fluid communication with the mouthpiece bore and a distal end,
   the device thereby configured to allow a person whose mouth sealingly contacts the portion of the external surface of the body that surrounds the opening to provide fluid communication between the person's mouth-hole and the opening to:
inhale a gas into the mouth, the gas first passing through the gas conduit, the mouthpiece bore, and then out through the opening of the mouthpiece into the mouth to be inhaled so that the device is an inhalation device;
exhale a gas from the mouth into the opening of the mouthpiece, through the mouthpiece bore into the gas conduit so that the device is an exhalation device; or
inhale a gas into the mouth, the gas first passing through the gas conduit, the mouthpiece bore, and then out through the opening of the mouthpiece into the mouth to be inhaled and exhale a gas from the mouth into the opening of the mouthpiece, through the mouthpiece bore into the gas conduit so that the device is an inhalation/exhalation device.

In preferred embodiments, the device is configured so that when a person's mouth sealingly contacts the portion of the external surface of the body that surrounds the opening to provide fluid communication between the person's mouth-hole and the opening, the opening is not inside the person's mouth.

In preferred embodiments, the mouthpiece is configured so that the portion of the external surface of the body that surrounds the opening rests against the front of the lips of the person and is not not inside the person's mouth.

In some embodiments, the body has a bottom, a front, a left side and a right side, and the mouthpiece is located near the bottom-front of the device so that during use when the mouth of a user sealingly contacts the portion of the external surface that surrounds the opening, the user's lower lip contacts the bottom of the device and the user's upper lip contacts the front of the device so that the opening is in fluid communication with the mouth-hole of the user.

In some preferred embodiments, the opening is located on the front of the device near the bottom-front of the device, as in the embodiments depicted in FIGS. 2, 3, 4 and 5. In some alternative embodiments, the opening is located at the bottom-front of the device.

In some embodiments, the bottom-front of the device includes a clearly-definable physical feature such as an edge, a corner or other discontinuity, for example, a discontinuity that constitutes the intersection of the front with the bottom as in the embodiments depicted in FIGS. 2, 3, 4 and 5. In some alternative embodiments, for example, when the device is such that there is a continuous curved transition from the front to the bottom, the bottom-front is not a clearly-defined physical feature but is a general area of the device that, as detailed above, allows the user's lower lip to contact the bottom of the device and the user's upper lip to contact the front of the device so that the opening of the mouthpiece is in fluid communication with the mouth-hole of the user.

In some embodiments, the device further comprises a top and a back in addition to the bottom, the front, the left side and the right side.

In some embodiments, the gas conduit extends from proximate to the bottom of the body upwards towards the top of the body.

In some embodiments, the front of the body is flat. Alternatively, in some embodiments the front of the body is outwardly curved (convex).

In some embodiments, the bottom of the body is flat. Alternatively, in some embodiments, the bottom of the body is outwardly curved (convex).

In some embodiments, the device is configured so that during use, a vertical axis of the device is at an angle relative to the vertical axis of the user's head, the angle being between 20° and 70°, more preferably between 35° and 55°, and even more preferably between 40° and 50°.

In some embodiments, the mouthpiece comprises a mouthpiece front, a mouthpiece bottom, a mouthpiece left side and a mouthpiece right side, wherein the opening of the mouthpiece is on the front of the mouthpiece, as in the embodiments depicted in FIGS. 2, 3, 4 and 5. In such embodiments, the mouthpiece front constitutes the body front or a component of the body front, the mouthpiece bottom constitutes the body bottom or a component of the body bottom, the mouthpiece left side constitutes the body left side or a component of the body left side and the mouthpiece right side constitutes the body right side or a component of the body right side.

In some embodiments, the width of the front and of the bottom of the mouthpiece is between about 15 mm and about 35 mm.

In some embodiments, the opening is flush with the front of the mouthpiece.

In some embodiments, the front of the mouthpiece is smooth and devoid of features that protrude from the surface by more than about 1 mm, more preferably by more than about 0.5 mm. In some alternative embodiments, the device further comprises one or more than one protrusion on the front of the mouthpiece that protrude no more than about 2 mm from the front. Such protrusions provide tactile feedback to the lips, allowing a person to correctly position the lips relative to the external surface of the device to ensure fluid communication with the opening.

In some embodiments, at the height of the opening, the front of the mouthpiece is convexly curved. In some such embodiments, the convex curvature of the front of the mouthpiece is such that, in side view, a depth of the opening is between about 2 mm and about 6 mm.

In some embodiments, the angle between the front and the bottom of the mouthpiece is greater than 70°, in some preferred embodiments the angle is between 90.5° and 110°, see FIG. 2G.

In some embodiments, the shape of the opening on the front of the mouthpiece is selected from the group consisting of a continuous closed curve, a circle, an oval and a polygon

In some embodiments, the shape of the opening on the front of the mouthpiece is such that the width of the opening is larger than the height of the opening.

In some embodiments, the height of the opening is between about 3 mm and about 10 mm.

In some embodiments, the width of the opening is between about 10 mm and about 25 mm.

In some embodiments, the distance of the center of the opening from the bottom of the mouthpiece is between about 5 mm and about 10 mm.

### Device Comprising Two Reversibly-Mateable Assemblies

According to an aspect of some embodiments of the teachings herein, there is also provided an inhalation and/or exhalation device, comprising:
two reversibly-mateable assemblies having a mated state and a separated state,
   a body assembly; and
   a conduit assembly comprising a mouthpiece and a gas conduit,
      the mouthpiece comprising a mouthpiece bore having an opening;
      in the mated state, the opening at an external surface of the device and the gas conduit having a proximal end in fluid communication with the mouthpiece bore and a distal end;
the device thereby configured to allow a person whose mouth sealingly encircles the opening to:
   inhale a gas into the mouth, the gas first passing through the gas conduit, the mouthpiece bore, and then out through the opening of the mouthpiece into the mouth to be inhaled so that the device is an inhalation device;
   exhale a gas from the mouth into the opening of the mouthpiece, through the mouthpiece bore into the gas conduit so that the device is an exhalation device; or
   inhale a gas into the mouth, the gas first passing through the gas conduit, the mouthpiece bore, and then out through the opening of the mouthpiece into the mouth to be inhaled and exhale a gas from the mouth into the opening of the mouthpiece, through the mouthpiece bore into the gas conduit so that the device is an inhalation/exhalation device.

In some embodiments, in the mated state the two assemblies are immovably mated.

In some embodiments, the body assembly comprises an electrical power source such as a battery. In some such embodiments, the body assembly comprises at least one component operable with electricity from the power source. In some embodiments, at least one of the at least one component is selected from the group consisting of: an exhalation determiner, a camera, a light emitter, a light detector, a controller (including an analogue controller and a digital controller, for instance having an integrated circuit), a user display (including a display screen such as an LCD or LED screen and also one or more display lights, typically LEDs), a vibration unit (for example, as known in the art of cellular telephones), an audio transducer or a speaker, a USB connector, wireless communication circuitry, and a sensor such as a humidity sensor, a flow meter or a velocity meter, a pump to move (e.g., inject) a material held in a reservoir associated with the device into the gas conduit for inhalation, a nebulizer to generate a mist from a material held in a reservoir associated with the device, which mist is generated in and/or directed into the gas conduit for inhalation, and a valve configured to allow passage of a material from a reservoir associated with the device into the gas conduit for inhalation.

Additionally or alternatively, in some embodiments, the conduit assembly comprises a component that requires electrical power to operate, for example, one or more components as recited above. In some such embodiments, the conduit assembly comprises a power source such as a battery to provide power to such a component. Such embodiments are less preferred as the presence of a power source such as a battery in the conduit assembly makes disposal thereof more challenging.

In some embodiments, the conduit assembly includes a component that must be in electrical communication with a component of the body assembly to operate (for example, the conduit assembly includes a component that requires electrical power from a power source of the body assembly) and the conduit assembly and the body assembly are together configured so that in the mated states, the component of the conduit assembly is in electrical communication with the component of the body assembly (e.g., the component of the conduit assembly receives electrical power from a power source of the body assembly). In some preferred embodiments, such configuration includes a set of electrical contacts on the body assembly functionally associated with the body assembly component and a matching set of electrical contacts on the conduit assembly functionally associated with the conduit assembly component, the two sets of electrical contacts being electrically connected when the two assemblies are in a mated stated so that one or more components of the conduit assembly are in electrical communication with one or more components of the body assembly, and the two sets of electrical contacts are disconnected when the two assembles are in a separated state.

In some embodiments, the conduit assembly is devoid of any component that requires electrical power to operate and any component that requires to be in electrical communication with a component of the body assembly: generally, such conduit assemblies are preferred because there are more suitable to be single-use, limited-number of uses and/or disposable.

In some embodiments, the conduit assembly is made of plastic.

In some embodiments, the conduit assembly is at least one of biodegradable, compostable and home-compostable.

The conduit assembly and the body assembly are held together in the mated state using any suitable component and/or configuration. In some embodiments, a body assembly comprises a conduit-assembly passage. In some such embodiments, the conduit-assembly passage extends from the bottom towards the top of the body assembly. In some embodiments, in the mated state, the gas conduit is at least partially contained inside the conduit-assembly passage. In some embodiments, the conduit assembly is configured to slidingly move into the conduit-assembly passage from the separated state to the mated state and slidingly moved out of the conduit-assembly passage from the mated state to the separated state. Alternatively, in some embodiments the body assembly comprises a conduit-assembly groove in which the conduit assembly rests in the mated state. Additionally or alternatively, in some embodiments, the body assembly comprises one or more releasable connectors that in the mated state engage a portion of the conduit assembly and/or the conduit assembly comprises one or more releasable connectors that in the mated state engage a portion of the body assembly.

In some embodiments, the two assemblies are held together in the mated state by frictional force and separating the two assemblies from the mated state to the separated state requires applying a force sufficient to overcome the frictional force. In some embodiments, the frictional force is such that a manual force applied by a 170 cm tall 60 kg healthy human male is sufficient to overcome the frictional force.

Additionally or alternatively to the frictional force, in some embodiments, the device further comprises a locking mechanism which locks the two assemblies together in the mated state and must be manipulated in order to unlock the locking mechanism, in order to allow separation of the two assemblies to the separated state.

In some such embodiments, a locking mechanism comprises matching screw threads on the two assemblies, wherein: in the mated state the screw threads are screwed together; and the manipulation of the locking mechanism comprises rotating at least one of the two assemblies relative to the other assembly so as to unscrew the screw threads, thereby separating the two assemblies to the separated state.

Additionally or alternatively, in some embodiments, a locking mechanism comprises matching components of a mechanical snap on the two assemblies, wherein: in the mated state the matching components of the mechanical snap are locked together; and the manipulation of the locking mechanism comprises applying a force to at least one of the component of the mechanical snap to deform a portion of the mechanical snap, thereby allowing separation of the two assemblies to the separated state.

Additionally or alternatively, in some embodiments, a locking mechanism comprises a locking slide on at least one of the two assemblies, wherein: in the mated state the locking slide is in a locked position, preventing separation of the two assemblies; and the manipulation of the locking mechanism comprises applying a force to move the locking slide to an unlocked position, thereby allowing separation of the two assemblies to the separated state.

Additionally or alternatively, in some embodiments, a locking mechanism comprises at least one magnet on a first one of the two assemblies, wherein: in the mated state the magnet applies a magnetic force to a component (e.g., another magnet, a ferromagnetic component) on the second one of the two assemblies; and the manipulation of the locking mechanism comprises applying a force sufficient to overcome the magnetic force, thereby allowing separation of the two assemblies to the separated state.

According to an aspect of the teachings herein, there is also provided a kit comprising a device having two reversibly-mateable assemblies as described above, the kit having at least two conduit assemblies; and a body assembly. In some such embodiments, at least two of the at least two conduit assemblies of the kit are identical. Additionally or alternatively, in some embodiments at least two of the at least two conduit assemblies of the kit are different.

In some embodiments, a device according to the teachings herein (having a non-protruding mouthpiece and/or two reversibly-mateable assemblies) is a handheld device configured and dimensioned to be handheld during use.

In some embodiments, the total weight of a device is not more than about 200 grams, not more than about 150 grams and even not more than about 100 grams. Typically, the total weight of the device is not less than 25 grams.

Additionally or alternatively, in some embodiments, the greatest linear dimension of a device is not more than about 20 cm, not more than about 15 cm and even not more than about 10 cm. Typically the greatest linear dimension of a device is not less than about 4 cm.

In some embodiments, a device according to the teachings herein (having a non-protruding mouthpiece and/or two reversibly-mateable assemblies) is an inhalation device or an inhalation/exhalation device and the mouthpiece is an inhalation inlet of the device configured to allow a person whose mouth sealingly encircles the opening to inhale a gas, the gas first passing through the conduit and then out through the mouthpiece into the mouth to be inhaled. In some such embodiments, the device is configured to deliver a substance to the person whose mouth encircles the opening and inhales a gas into the mouth, the substance carried by the inhaled gas. In some such embodiments, the substance comprises, consists essentially or consists of a medicament. In some such embodiments the substance is selected from the group consisting of: a gaseous medicament; a liquid medicament present in the gas as droplets and/or an aerosol, a dry-powder drug so that the device constitutes a dry-powder inhaler, nicotine, a cannabinoid (such as CBD and/or THC), a flavonoid such as a flavone or a flavanol, a medicament for absorption through the oral mucosa, a medicament suitable for pulmonary administration, a medicament for treatment of a lung disorder; and a medicament for treatment of asthma.

In some embodiments, a device according to the teachings herein (having a non-protruding mouthpiece and/or two reversibly-mateable assemblies) is an exhalation device or an inhalation/exhalation device and the mouthpiece is an exhalation inlet configured to allow a person whose mouth sealingly encircles the opening to exhale a gas from the mouth, through the mouthpiece into the gas conduit. In some such embodiments, the device is a breathing biofeedback device, for example, a breathing biofeedback device according to the teachings of US 2015/0011906 A1.

A breathing biofeedback device includes an exhalation determiner which is a component that determines when breath is exhaled by a user into the opening of the mouthpiece, and in some embodiments also other parameters of exhaled breath such as exhalation velocity and/or volume.

Any suitable exhalation determiner may be used for implementing a device according to the teachings herein, including an exhalation determiner described in US 2015/0011906. Exhalation determiners described in US 2015/0011906 include:
an exhalation determiner that comprises a component such as a propeller that rotates as a result of exhaled breath, the rotating propellor generating electricity that is considered a signal indicating exhalation;
an exhalation determiner that comprises an at least partially conductive component such as a ball that ordinarily rests on two electrical contacts to conduct electricity therebetween which is lifted from the electrical contacts as a result of exhaled breath, where the lack of electricity passing through the component between the two contacts is considered a signal indicating exhalation;
an exhalation determiner that comprises a component that is biased to a first position and is moved to a second position as a result of exhaled breath, where in the second position light is blocked from reaching a detector, where the lack of light reaching the detector is considered a signal indicating exhalation; and
an exhalation determiner that comprises a component that is biased to a first position and is moved to a second position as a result of exhaled breath, where in the second position an electrical circuit is closed allowing electricity to pass through a circuit, where electricity passing through the circuit is considered a signal indicating exhalation.

In some embodiments, a device according to the teachings herein comprises two separable assemblies as described above (with or without a non-protruding mouthpiece) and an exhalation determiner, the exhalation determiner comprising a first component which is part of the conduit assembly and a second component which is part of the body assembly, wherein operation of the exhalation determiner requires electrical communication between the first component and the second component, and the conduit assembly and the body assembly are together configured so that in the mated states, the first component and the second component are in electrical communication, and in the separated state the first component and the second component are not in electrical communication. In some preferred embodiments, such configuration includes a set of electrical contacts on the body assembly functionally associated with the body assembly component of the exhalation determiner and a matching set of electrical contacts on the conduit assembly functionally associated with the conduit assembly component of the exhalation determiner, the two sets of electrical contacts being electrical connected when the two assemblies are is in a mated stated so that the two components of the exhalation determiner are in electrical communication, and the two sets of electrical contacts are disconnected when the two assembles are in a separated state.

In some embodiments, a device according to the teachings herein comprises two separable assemblies as described above (with or without a non-protruding mouthpiece) and an exhalation determiner, the exhalation determiner comprising a first component which is part of the conduit assembly and a second component which is part of the body assembly, wherein operation of the exhalation determiner does not require electrical communication between the first component and the second component. In some such embodiments:
a moveable component of the exhalation determiner that is associated with the conduit assembly configured to move from a rest position to a different position in response to exhalation into the mouthpiece; and
a detection component of the exhalation determiner that is associated with the body assembly that includes an optical detector that is configured in the mated state of the two assemblies to differentiate between the rest position and the different position of the moveable component. In some such embodiments, at least a portion of the conduit assembly is transparent. Some such embodiments are substantially similar to the exhalation determiner depicted in FIGS. 5A and 5B of US 2015/0011906, where the diaphragm is disposed inside the conduit of the conduit assembly and the laser and light detector are components of the body assembly.

Aspects and embodiments of the invention are described in the description hereinbelow and/or in the appended figures and claims.

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale. In the Figures:

FIGS. 1A and 1B (prior art) schematically depict a CalmiGo® breathing biofeedback device in a side cross section (FIG. 1A) and in a side view (FIG. 1B); and

FIGS. 2A-2G schematically depict an embodiment of an exhalation device according to the teachings herein in: FIG. 2A (side cross section, fully assembled), FIG. 2B (side cross section, separated state), FIG. 2C (perspective view, from the front), FIG. 2D (side view), FIG. 2E (mouthpiece, front view), and FIGS. 2F and 2G (mouthpiece, side view);

FIG. 3 schematically depicts an embodiment of an exhalation device in side view;

FIG. 4 schematically depicts an embodiment of an inhalation device in side cross section;

FIG. 5 schematically depicts an embodiment of an inhalation/exhalation device in side cross section; and

FIGS. 6A and 6B schematically depict an embodiment of an exhalation device in side cross section.

### DESCRIPTION OF SOME EMBODIMENTS OF THE INVENTION

The invention, in some embodiments, relates to inhalation and exhalation devices. In some embodiments the disclosed devices have a mouthpiece, preferably a non-protruding mouthpiece. In some embodiments, the devices comprise two assemblies that are reversibly mateable, one of the two assemblies comprising a conduit for the passage of air.

The principles, uses and implementations of the teachings of the invention may be better understood with reference to the accompanying description and figures. Upon perusal of the description and figures present herein, one skilled in the art is able to implement the teachings of the invention without undue effort or experimentation. In the figures, like reference numerals refer to like parts throughout.

Before explaining at least one embodiment of the invention in detail, it is to be understood that the invention is not limited in its application to the details of construction and the arrangement of the components and/or methods set forth herein. The invention is capable of other embodiments or of being practiced or carried out in various ways. The phraseology and terminology employed herein are for descriptive purpose and should not be regarded as limiting.

Different aspects of the teachings herein will be exemplified with an embodiment of an exhalation device, specifically a breathing biofeedback device.

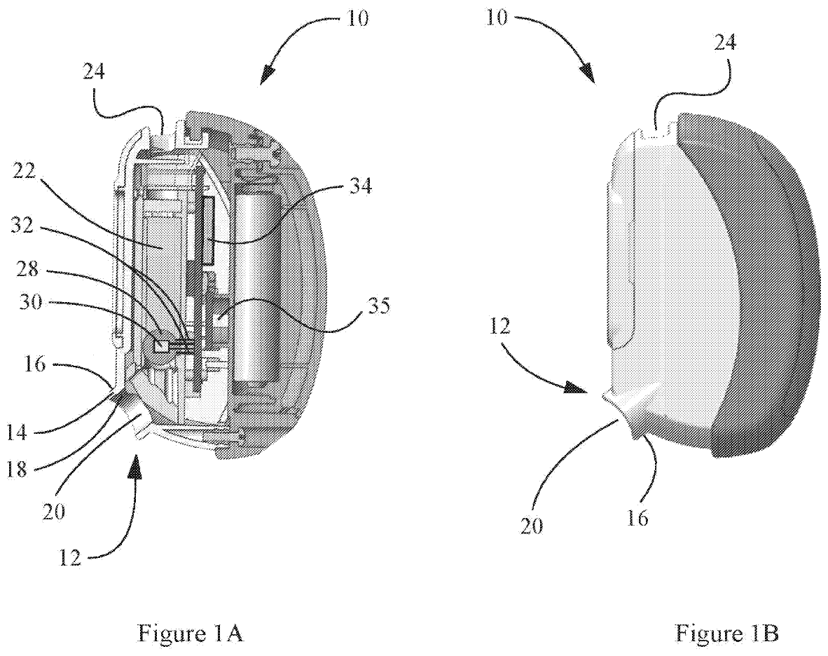

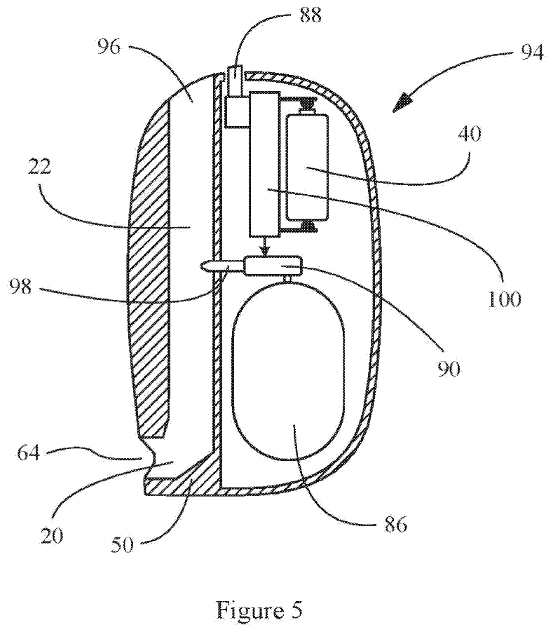

In US 2015/0011906 are described exhalation devices that are breathing biofeedback devices to assist a person in proper breathing. An embodiment of such exhalation device is commercially available from Dendro Technologies, Inc. (New York, NY, USA) as CalmiGo®. Such a device 10 is schematically depicted in a side cross section in FIGS. 1A and 1n a side view in FIG. 1B. Device 10 includes a protruding hollow mouthpiece 12 that constitutes the exhalation inlet of device 10. Mouthpiece 12 is defined by a wall 14 having an outer wall surface 16 and an inner wall surface 18 defining a mouthpiece bore 20 with a roughly oval cross section. Bore 20 is in fluid communication with a tubular exhalation conduit 22 which distal end 24 constitutes the exhalation outlet of device 10. Device 10 includes an exhalation determiner that operates in a manner analogous to the device disclosed in US 2015/0011906 in FIG. 5 thereof. Specifically, at the intersection of mouthpiece bore 20 and exhalation conduit 22 is a moveable component 28, which is a component of the exhalation determiner. Additional components of the exhalation determiner include a LED laser 30 and a light detector 32. In FIG. 1, the body of light detector 32 is hidden from view by moveable component 28 and only the connecting pins of light detector 32 are depicted in FIG. 1A).

When device 10 is activated and oriented for use (preferably with the axis of conduit 22 within 30° of perpendicular with the gravity vector and distal end 24 being higher than mouthpiece 12), moveable component 28 is biased to a first position. In the first position, light from LED laser 30 is blocked from reaching light detector 32. A controller 34 (an integrated circuit with computer processor on a populated circuit board, PCB) registers the absence of a signal received from light detector 32 as indicating that no air exhaled by a user into mouthpiece 12 is passing through exhalation conduit 22.

During use of device 10, a user encircles outer wall surface 16 with the lips to make a seal so that the opening of the mouthpiece bore is inside the user's mouth. The user then exhales through the mouth. The exhaled breath passes through mouthpiece bore 20, moving moveable component 28 to a second position. When the moveable component 28 is in the second position, light from laser 30 reaches light detector 32. Controller 34 receives a signal from light detector 32 that light from laser 30 is detected as indicating that air exhaled by a user into mouthpiece 12 is passing through exhalation conduit 22. The duration of the signal received from light detector 32 by controller 34 is considered the exhalation duration. Device 10 further comprises a vibration unit 35 as known in the art of smartphones and, in some embodiments, is used to provide tactile feedback to a user.

Exhalation Device with Replaceable Exhalation Conduit

A disadvantage of a device such as device 10 is that such a device is preferably used by a single individual for hygienic reasons.

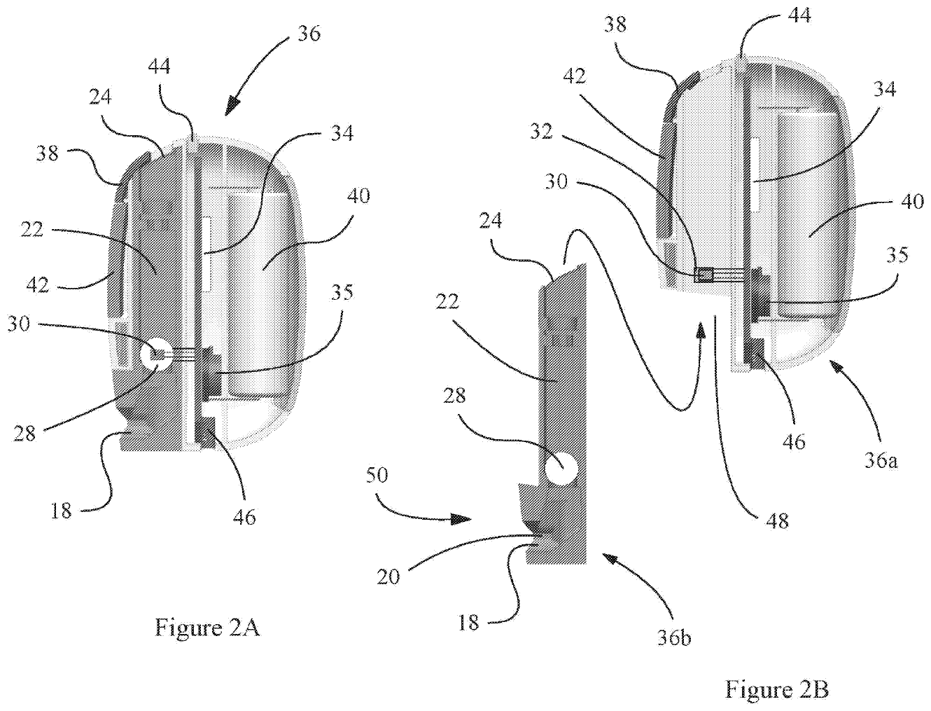

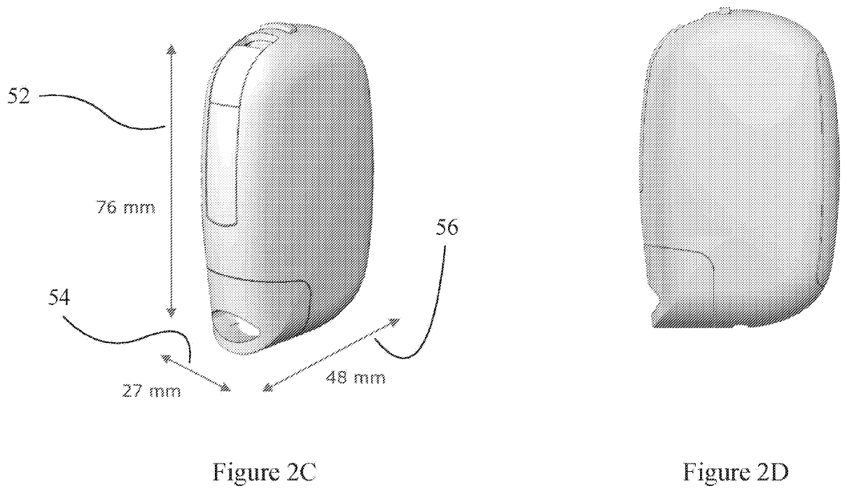

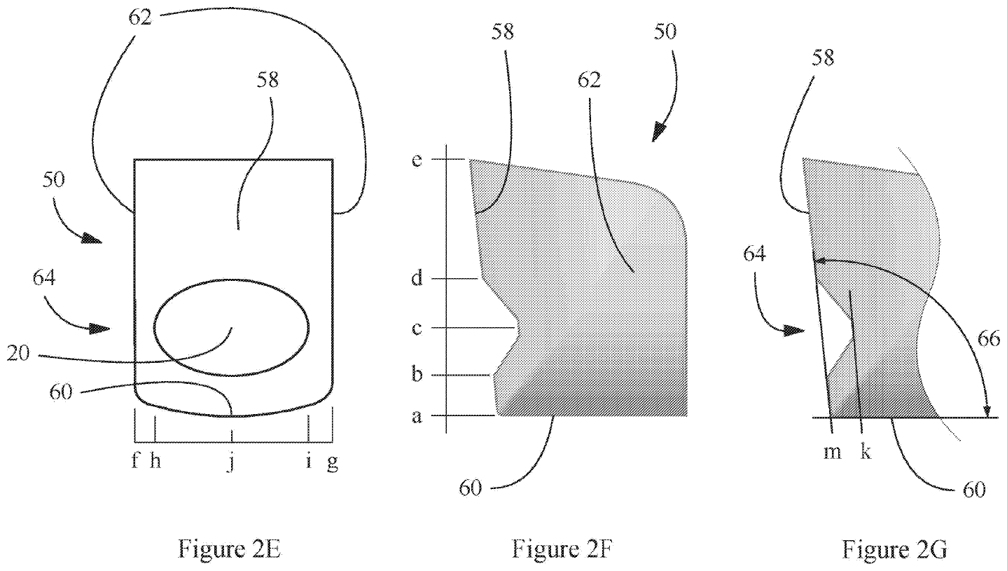

An exhalation device devoid of these potential problems is a breathing biofeedback device 36 schematically depicted in:
FIG. 2A (side cross section, fully assembled);
FIG. 2B (side cross section, separated state);
FIG. 2C (perspective view, from the front);
FIG. 2D (side view);
FIG. 2E (mouthpiece, front view); and
FIGS. 2F and 2G (mouthpiece, side view).

Device 36 is substantially similar in construction and operation to device 10 and shares many of the same or equivalent components.

An aspect of some embodiments of the teachings herein as embodied in device 36 is that the device comprises two assemblies: a body assembly 36a and an exhalation conduit assembly 36b which can be reversibly mated. In FIG. 2A, the two assemblies are depicted mated and in FIG. 2B the two assemblies are depicted separated one from the other.

Body assembly 36a includes components such as a LED laser 30 and a functionally-associated light detector 32, a controller 34, LED indicators or a user display 38 to function as a reporter to provide a user with a visual signal related to an exhalation duration, battery 40 as a power supply, an inhalable substance outlet and dispenser for emitting configured for emitting a scent 42, an On/Off switch 44, a vibration unit 35 and a USB connector 46 allowing electronic communication between controller 34 and an external device such as a computer or smartphone and/or for charging of battery 40. Body assembly 36a also includes a conduit-assembly passage 48 extending from the bottom to the top of body assembly 36a. In some embodiments, body assembly 36a and conduit assembly 36b are locked together by a locking mechanism. Such a mechanism is not depicted in device 36. Depending on the specific embodiments, a suitable such locking mechanism may include one or more of matching screw threads, mechanical snaps, a locking slide, a magnet on one or both assemblies and combinations thereof.

Exhalation conduit assembly 36b includes a non-protruding mouthpiece 50 that constitutes the exhalation inlet of device 36 with a mouthpiece bore 20, a tubular exhalation conduit 22 made of plastic transparent to light from laser 30, and which distal end 24 constitutes the exhalation outlet of device 36, and a moveable component 28 confined inside exhalation conduit 22.

Moveable component 28, laser 30 and light detector 32 are components of an exhalation determiner of device 36.

The outer dimensions and shape of exhalation conduit assembly 36b are such that exhalation conduit assembly 36b and body assembly 36a can be slidingly mated by pushing exhalation conduit assembly 36b into conduit-assembly passage 48 from the bottom of body assembly 36a. When exhalation conduit assembly 36b is fully inserted into body assembly 36a, exhalation conduit assembly 36b and conduit-assembly passage 48 immovably mate by a combination of friction and/or the action of a locking mechanism (if present) and require a physical effort and/or unlocking of the locking mechanism (if present) to be separated.

When device 36 is held for use, component 28 is inside exhalation conduit 22 in a first position relative to laser 30 and light detector 32 analogous to the described for device 10 allowing the use of device 36 in a manner analogous to the use of device 10. The action of device 36 is analogous to that of device 10 as well.

If desired, a user can separate exhalation conduit assembly 36b from body assembly 36a by grasping mouthpiece 50 with the fingers of one hand while holding body assembly 36a with a second hand and pulling firmly. In embodiments comprising a locking mechanism, such separation may involve other and/or additional steps, for example, unlocking the locking mechanism. Subsequently, exhalation conduit assembly 36b is optionally cleaned, for example by one or more of rinsed, washed, disinfected and autoclaved. Optionally, a different exhalation conduit assembly is mated with body assembly 36a. Such a different exhalation conduit assembly may be identical to the original exhalation conduit assembly 36b, or may be different, for example, the bore of the different exhalation mouthpiece may have different dimensions more suited for a specific user or for particular users' category.

In preferred embodiments, exhalation conduit assembly 36b and its components are made of inexpensive plastic parts (including moveable component 28) so that exhalation conduit assembly 36b can be single-use and/or disposable and/or biodegradable (even compostable and even home compostable). Body assembly 36a which includes relatively expensive components such as laser 30, light detector 32, controller 34 and battery 40 is preferably reused.

Cleaning or replacing exhalation conduit assembly 36b allows a body assembly 36b to be used by multiple users.

Device with Non-Protruding Mouthpiece

Prior art inhalation and exhalation devices such as breathing biofeedback device 10 or prior art inhalers include a protruding hollow mouthpiece which is substantially a tube having an outer wall surface and an inner wall surface defining a bore.

For use, a person encircles the protruding outer wall with the lips to make a seal so that the opening of the bore is inside the person's mouth, and then inhales or exhales. A protruding mouthpiece is relatively expensive to manufacture, is somewhat inconvenient to store in a pocket or handbag and is relatively susceptible to damage.

An aspect of some embodiments of the teachings herein as embodied in device 36 is that the device comprises a non-protruding mouthpiece 50. The Inventors have discovered that it is possible to provide an inhalation device, exhalation device or inhalation/exhalation device that includes a non-protruding mouthpiece which mouthpiece is configured in a way that allows a user to intuitively hold the device in a way that the mouthpiece is oriented with respect to the mouth in a way that allows inhalation and/or exhalation thereinto for an extended period of time without inconvenience and with substantially greater comfort.

The device is provided with a body comprising an external surface and a non-protruding mouthpiece having a mouthpiece bore with an opening at the external surface. The device is configured to allow a person whose mouth sealingly contacts the portion of the external surface of the body that surrounds the opening to provide fluid communication between the mouth-hole and the opening to exhale from the mouth into the opening or to inhale from the opening into the mouth.

In some embodiments, the opening of the mouthpiece is located on a front of the device, near the bottom of the device, e.g., near a bottom-front corner of the device. During use, a user's lower lip contacts the bottom of the device and the user's upper lip contacts the front of the device so that the opening of the device is in fluid communication with the mouth hole of the user. In such a way, the bottom front corner (defined by the intersection of the front and the bottom of the device) functions in a manner analogous to prior protruding mouthpieces by providing a physical part of the device around which the lips form a seal while the opening of the device is located inside the mouth hole of the user, the geometry of the bottom front corner, where the shape and dimensions of the front and the bottom of the device together determine the orientation of the device relative to the face and head of the user. In the device according to the teachings herein, the vertical axis of the device (an imaginary axis parallel to the front apex or front face of the device) is angled relative to the vertical axis of the user's head (the axis defined by the intersection of the sagittal plane and the coronal plane of the head). Preferably the angle is between 20° and 70°, more preferably between 35° and 55°, and even more preferably between 40° and 50°. Such angles allow the device to be used intuitively in a comfortable fashion for extended period of time, and allow formation of an effective seal by the lips around the opening of the device. In some embodiments, the front is flat, but in alternative preferred embodiments is outwardly curved (convex). In some embodiments the bottom is flat but in alternative preferred embodiments is outwardly curved (convex).

Breathing biofeedback devices such as 36 are configured to be compact so as to be easily carried around, by a person, in some embodiments having a height dimension 52 of 60-90 mm, a width dimension 54 of 20-35 mm and a depth dimension 56 of 40-60 mm. Device 36 depicted in FIG. 2 has a height dimension 52 of 76 mm, a width dimension 54 of 27 mm and a depth dimension 56 of 48 mm.

In some embodiments, a non-protruding mouthpiece according to the teachings herein comprises a front, a bottom and two sides (left side and right side), where on the front is an opening in fluid communication with the bore of the device. In inhalation devices, the opening is the outlet of the device. In exhalation devices, the opening is the inlet of the device. An embodiment of a non-protruding mouthpiece 50 is depicted in FIGS. 2A-2G, but in particular detail in FIG. 2E (front view) and FIGS. 2F and 2G (side view). Non-protruding mouthpiece 50 comprises a front 58, a bottom 60, two sides 62, and an opening 64 (which in device 36 is an exhalation inlet) in fluid communication with bore 20.

As seen in FIGS. 2A-2G, in a mouthpiece according to the teachings herein, the opening is flush with the front of the mouthpiece. In some non-depicted embodiments, the front has one or more protrusions that protrude no more than about 2 mm from the front, in some embodiments surrounding the opening allowing a person's lip to tactilely register the protrusions in order to place the lips properly in relation to the opening.

In some embodiments, at the height of the opening, the front is convexly curved. In FIG. 2F is seen that at the height of opening 64 (from b to d), front 58 is convexly curved. As a result, in side view (such as depicted in FIGS. 2F and 2G), the opening appears as a notch in the front. The convex curvature of the front is any suitable curvature. In some embodiments, the curvature is such that in side view, the depth of the notch corresponding to the opening is between about 2 mm and about 6 mm. As seen in FIG. 2G, in mouthpiece 50 the depth of the notch corresponding to opening 64 (k-m) is 2.5 mm.

An angle between the front and the bottom is any suitable angle greater than 70°; in preferred embodiments the angle is between 90.5° and 110°. As seen in FIG. 2G, in mouthpiece 50 an angle 66 between front 58 and bottom 60 is 96°.

The shape of the opening on the front is any suitable closed curve, preferably a continuous closed curve such as a circle or an oval. In preferred embodiments, the width of the opening (h-i) is larger than the height of the opening (b-d).

The width (f-g) of the front 58 and the bottom 60 is any suitable width, preferably between about 15 mm and about 35 mm. In mouthpiece 50 depicted in FIGS. 2, the width (f-g) of front 58 and bottom 60 is 25 mm.

The height of the opening (b-d) is any suitable height, preferably between about 3 mm and about 10 mm. In mouthpiece 50 depicted in FIGS. 2, the height of opening 64 is 7.5 mm.

The width of the opening (h-i) is any suitable width, preferably between about 10 mm and about 25 mm. In mouthpiece 50 depicted in FIGS. 2, the width of opening 64 is 16 mm. The dimensions of the opening such as 64 are selected to ensure proper airflow during inhaling and/or exhaling in accordance with the intended use of the specific device.

The distance of the center of the opening from the bottom of the mouthpiece (a-c) is any suitable height, preferably between about 5 mm and about 10 mm. In mouthpiece 50 depicted in FIGS. 2, the height of the center of opening 64 from bottom 60 is 7 mm.

The invention has been specifically described with reference to an embodiment of an exhalation device, device 36. The teachings herein are applicable and in some embodiments are implemented with an inhalation device or an inhalation/exhalation device.

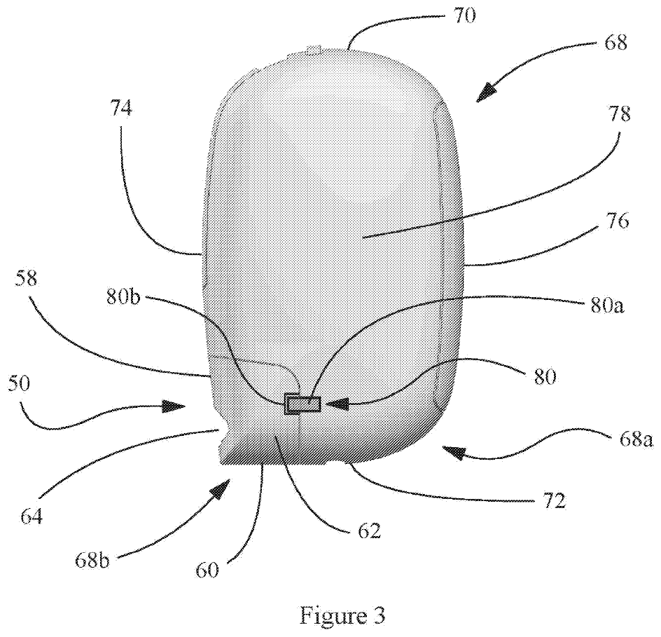

In FIG. 3 is schematically depicted an additional embodiment of an exhalation device according to the teachings herein, device 68, in side view. Device 68 is substantially the same as exhalation device 36 depicted in FIGS. 2A-2G and like device 36 comprises a body assembly 68a and a conduit assembly 68b and also includes a top 70, a bottom 72, a front 74, a back 76, a right side 78 and a left side (located opposite right side 78, so is not depicted in FIG. 3). Like in conduit assembly 68b of device 36, conduit assembly 68b of device 68 includes a non-protruding mouthpiece 50 according to the teachings herein, having a mouthpiece front 58, a mouthpiece bottom 60, two mouthpiece sides 62 (one depicted in FIG. 3) and an opening 64. Device 68 further comprises a locking mechanism 80 which locks body assembly 68a and conduit assembly 68b together in the mated state depicted in FIG. 3. To separate assemblies 68a and 68b to the separated state (analogous to the depicted in FIG. 2B), locking mechanism 80 must be unlocked by manipulating locking mechanism 80. Specifically, locking mechanism 80 comprises a locking slide 80a that is secured to the surface of body assembly 68a which, in the locked state is biased forward into a locking slot 80b on the surface of conduit assembly 68b. A user manipulates locking mechanism 80 by sliding locking slide 80a towards back 76 out of locking slot 80b, thereby unlocking locking mechanism 80.

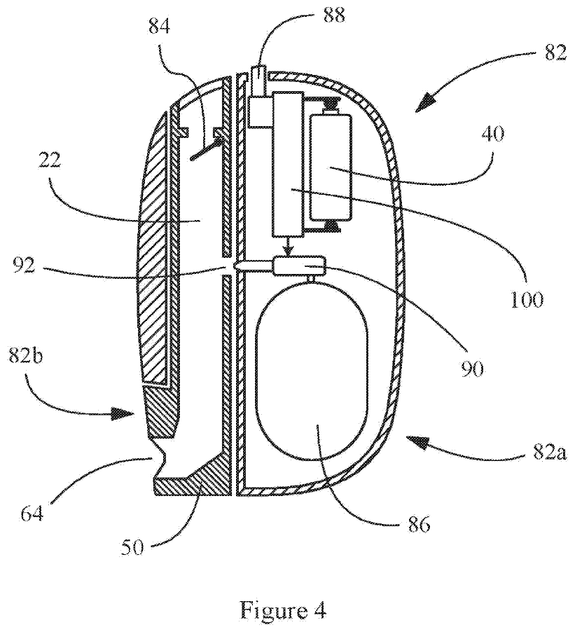

In FIG. 4 is schematically depicted an inhalation device according to the teachings herein, device 82, in side cross section. Device 82 comprises a body assembly 82a and a conduit assembly 82b. Conduit assembly 82b comprises, in addition to a non-protruding mouthpiece 50 according to the teachings herein and a gas conduit 22, a one-way valve 84 functionally associated with gas conduit 22 which allows inhalation of ambient air and gas through a gas conduit 22 but prevents exhalation therethrough. Body assembly 82a comprises a reservoir 86 which holds a liquid that can be inhaled when nebulized to form a mist. When a switch 88 is depressed, a pump/nebulizer 90 provided with power from a battery 40 under control of a controller 100, draws liquid from reservoir 86, and produces a mist therefrom that is introduced into gas conduit 22 through a gap 92 in the side of conduit assembly 82b. A person using device 82 can inhale the produced mist together with air into the mouth and optionally into the lungs through an opening 64 of a mouthpiece 50 but cannot exhale through the mouth into gas conduit 22 due to the closing of valve 84.

In FIG. 5 is schematically depicted an embodiment of an inhalation/exhalation device according to the teachings herein, device 94, in side cross section. Device 94 is not separable into two assemblies. Device 94 comprises a non-protruding mouthpiece 50 in accordance with the teachings herein having an opening 64 and a mouthpiece bore 20 in fluid communication with a gas conduit 22 which has a distal end 96 that is open to the ambient air. Device 94 comprises a reservoir 86 which holds a liquid that can be inhaled when vaporized to form a gas. When a switch 88 is depressed, a pump/vaporizer 90 provided with power from a battery 40 under control of a controller 100, draws liquid from reservoir 86, and vaporizes the liquid to be a gas that is introduced into gas conduit 22 through pipe 98. A person using device 94 can inhale the produced gas together with air that is drawn into gas conduit 22 through distal end 96 into the mouth and optionally into the lungs through opening 64 of mouthpiece 50. A person using device 94 can also exhale gas from the mouth, into opening 64, mouthpiece bore 20, gas conduit 22 and out through distal end 96 of gas conduit 22.

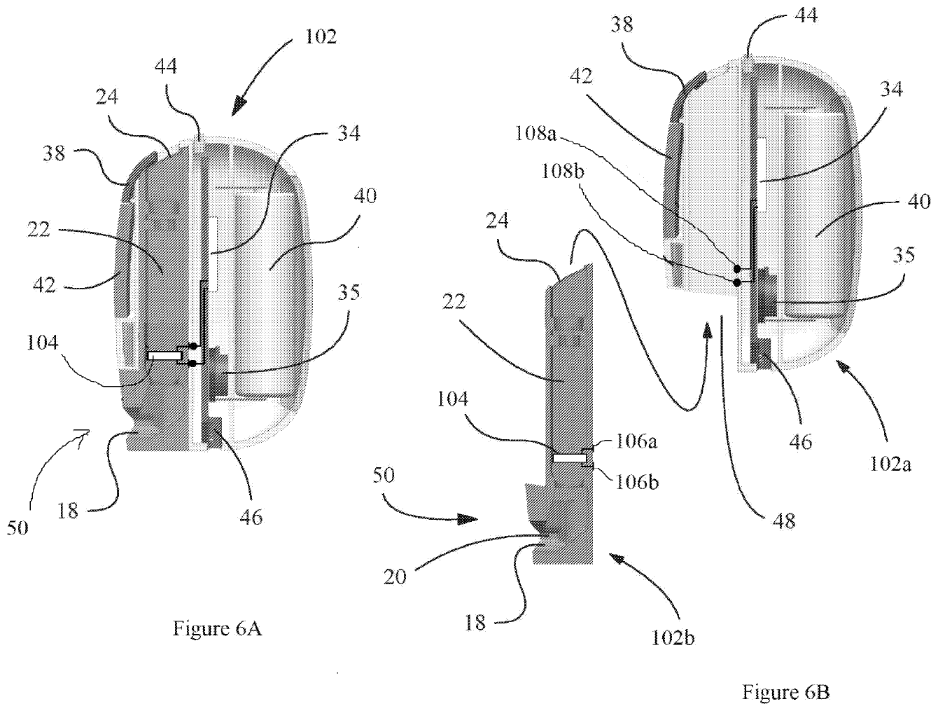

In FIGS. 6A and 6B is schematically depicted an exhalation device according to the teachings herein, device 102, in side cross section. Device 102 comprises a body assembly 102a and a conduit assembly 102b. Device 102 is a breathing biofeedback device and therefore conduit assembly 102b comprises, in addition to a non-protruding mouthpiece 50 according to the teachings herein and a gas conduit 22, an exhalation determiner 104 that requires electrical communication with a controller 34 to operate. Accordingly, conduit assembly 102b comprises two metal contacts 106a and 106b in electrical communication with exhalation determiner 104 while body assembly 102a comprises two contacts 108a and 108b in electrical communication with controller 34, contacts 108a and 108b being spring-loaded to be biased to protrude out into conduit-assembly passage 48. In the assembled state, the position of conduit assembly 102b inside conduit-assembly passage 48 of body assembly 102a is such that contacts 108a and 108b press against and therefore make robust electrical contact with contacts 106a and 106b. As a result, in the assembled state, there is electrical communication between exhalation determiner 104 and controller 34.

Unless otherwise defined, all technical and scientific terms used herein have the same meaning as commonly understood by one of ordinary skill in the art to which the invention pertains. In case of conflict, the specification, including definitions, takes precedence.

As used herein, the terms “comprising”, “including”, “having” and grammatical variants thereof are to be taken as specifying the stated features, integers, steps or components but do not preclude the addition of one or more additional features, integers, steps, components or groups thereof. As used herein, the indefinite articles “a” and “an” mean “at least one” or “one or more” unless the context clearly dictates otherwise.

As used herein, when a numerical value is preceded by the term “about”, the term “about” is intended to indicate +/−10%. As used herein, a phrase in the form “A and/or B” means a selection from the group consisting of (A), (B) or (A and B). As used herein, a phrase in the form “at least one of A, B and C” means a selection from the group consisting of (A), (B), (C), (A and B), (A and C), (B and C) or (A and B and C).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

Section headings are used herein to ease understanding of the specification and should not be construed as necessarily limiting.

1. An inhalation and/or exhalation device (36, 68, 82, 94) comprising:
a body comprising an external surface;
a non-protruding mouthpiece (50) comprising a mouthpiece bore (20) having an opening (64) at said external surface; and
a gas conduit (22) having a proximal end in fluid communication with said mouthpiece bore (20) and a distal end,
the device thereby configured to allow a person whose mouth sealingly contacts a portion of said external surface of said body that surrounds said opening to provide fluid communication between the mouth-hole of the person and said opening to:
inhale a gas into the mouth, the gas first passing through said gas conduit (22), said mouthpiece bore (20), and then out through said opening (64) of said mouthpiece (50) into the mouth to be inhaled so that the device is an inhalation device;
exhale a gas from the mouth into said opening (64) of said mouthpiece (50), through said mouthpiece bore (20) into said gas conduit (22) so that the device is an exhalation device; or
inhale a gas into the mouth, the gas first passing through said gas conduit (22), said mouthpiece bore (20), and then out through said opening (64) of said mouthpiece (50) into the mouth to be inhaled and exhale a gas from the mouth into said opening (64) of said mouthpiece (50), through said mouthpiece bore (20) into said gas conduit (22) so that the device is an inhalation/exhalation device.

2. The device of claim 1, wherein:
said body has a bottom (72), a front (74), a left side and a right side (78); and
said mouthpiece is located near a bottom-front of the device so that during use when the mouth of a user sealingly encircles said opening, the user's lower lip contacts said bottom of the device and the user's upper lip contacts said front of the device so that said opening of said mouthpiece is in fluid communication with the mouth of the user.

3. The device of claim 2, wherein said gas conduit extends from proximate to said bottom of said body upwards towards said top of said body.

4. The device of any one of claims 2 to 3, wherein said front of said body is outwardly curved.

5. The device of any one of claims 2 to 3, wherein said front of said body is flat.

6. The device of any one of claims 2 to 5, wherein said bottom of said body is outwardly curved.

7. The device of any one of claims 2 to 5, wherein said bottom of said body is flat.

8. The device of any one of claims 1 to 7, wherein the device is configured so that during use, a vertical axis of the device is at an angle relative to the vertical axis of the user's head, said angle being between 20° and 70°.

9. The device of any one of claims 1 to 8, wherein said mouthpiece (50) comprises a front (58), a bottom (60), a left and a right side (62) wherein said opening (64) of said mouthpiece (50) is on said front (58) of said mouthpiece (50).

10. The device of claim 9, wherein a width of said front (58) and of said bottom (60) of said mouthpiece is between about 15 mm and about 35 mm.

11. The device of any one of claims 9 to 10, wherein said opening (64) is flush with said front (58) of said mouthpiece (50).

12. The device of any one of claims 9 to 11, wherein at the height of said opening (64), said front (58) of said mouthpiece (50) is convexly curved.

13. The device of claim 12, wherein a convex curvature of said front (58) of said mouthpiece is such that, in side view, a depth of said opening (64) is between about 2 mm and about 6 mm.

14. The device of any one of claims 9 to 13, wherein an angle (66) between said front (58) and said bottom (60) of said mouthpiece is greater than 70°.

15. The device of any one of claims 9 to 14, where a shape of said opening (64) on said front (58) is selected from the group consisting of a continuous closed curve, a circle and an oval and a polygon.

16. The device of any one of claims 9 to 15, where a shape of said opening (64) on said front (58) is such that a width of said opening (64) is larger than a height of said opening (64).

17. The device of any one of claims 9 to 16, wherein a height of said opening (64) is between about 3 mm and about 10 mm.

18. The device of any one of claims 9 to 17, wherein a width of said opening (64) is between about 10 mm and about 25 mm.

19. The device of any one of claims 9 to 18, wherein a distance of a center of said opening (64) from said bottom (64) of said mouthpiece (50) is between about 5 mm and about 10 mm.

20. An inhalation and/or exhalation device (36, 68, 82) comprising:
two reversibly-mateable assemblies (36a and 36b, 68a and 68b, 82a and 82b) having a mated state and a separated state,
a body assembly (36a, 68a, 82a); and
a conduit assembly (36b, 68b, 82b) comprising a mouthpiece (50) and a gas conduit (22), said mouthpiece (50 comprising a mouthpiece bore (20) having an opening (64);
in said mated state, said opening at an external surface of the device and said gas conduit (22) having a proximal end in fluid communication with said mouthpiece bore (20) and a distal end,
the device thereby configured to allow a person whose mouth sealingly encircles said opening (64) to:
inhale a gas into the mouth, the gas first passing through said gas conduit (20), said mouthpiece bore (20), and then out through said opening (64) of said mouthpiece (50) into the mouth to be inhaled so that the device is an inhalation device;
exhale a gas from the mouth into said opening (64) of said mouthpiece (50), through said mouthpiece bore (20) into said gas conduit (22) so that the device is an exhalation device; or
inhale a gas into the mouth, the gas first passing through said gas conduit (20), said mouthpiece bore (20), and then out through said opening (64) of said mouthpiece (50) into the mouth to be inhaled and exhale a gas from the mouth into said opening (64) of said mouthpiece (50), through said mouthpiece bore (20) into said gas conduit (22) so that the device is an inhalation/exhalation device.

21. The device of claim 20, wherein said mouthpiece is a non-protruding mouthpiece.

22. The device of any one of claims 20 to 21 wherein said conduit assembly (36b, 68b, 82b) is devoid of any component that requires electrical power to operate.

23. The device of any one of claims 20 to 22, wherein said body assembly (36a, 68a, 82a) comprises an electrical power source (40).

24. The device of claim 23 wherein said body assembly comprises at least one component (30, 32, 34, 35, 38, 46) operable with electricity from said electrical power source (40).

25. The device of any one of claims 20 to 24, wherein said conduit assembly is configured to slidingly move into a conduit-assembly passage (48) of said body assembly from said separated state to said mated state and slidingly moved out of said conduit-assembly passage (48) from said mated state to said separated state.

26. The device of any one of claims 20 to 25, wherein said two assemblies are held together in said mated state by frictional force and separating said two assemblies from said mated state to said separated state requires applying a force sufficient to overcome said frictional force.

27. The device of any one of claims 20 to 26, the device further comprising a locking mechanism which locks said two assemblies together in said mated state and must be manipulated in order to unlock said locking mechanism, in order to allow separation of said two assemblies to said separated state.

28. A kit comprising a device of any one of claims 20 to 27, comprising:
a body assembly (36a, 68a, 82a); and
at least two said conduit assemblies (36b, 68b, 82b).

29. The device of any one of claims 1 to 28, wherein the device is a handheld device configured and dimensioned to be handheld during use.

30. The device of any one of claims 1 to 29, wherein said device is an exhalation device or an inhalation/exhalation device and said mouthpiece (50) is an exhalation inlet configured to allow a person whose mouth sealingly encircles said opening (64) to exhale a gas from the mouth, through said mouthpiece into said gas conduit (20).

31. The device of any one of claims 1 to 29, wherein the device is an inhalation device or an inhalation/exhalation device and said mouthpiece is an inhalation inlet of the device configured to allow a person whose mouth sealingly encircles said opening to inhale a gas, the gas first passing through said conduit and then out through said mouthpiece into the mouth to be inhaled.

32. The device of claim 31, configured to deliver a substance to the person whose mouth encircles said opening and inhales a gas into the mouth, the substance carried by the inhaled gas.

33. The device of claim 32, wherein said substance comprises a medicament.

34. The device of any one of claims 32 to 33, wherein said substance is selected from the group consisting of: a gaseous medicament; a liquid medicament present in said gas as droplets and/or an aerosol; a dry-powder drug so that the device constitutes a dry-powder inhaler; nicotine; a cannabinoid; a flavonoid; a medicament for absorption through the oral mucosa; a medicament suitable for pulmonary administration; a medicament for treatment of a lung disorder and a medicament for treatment of asthma.
